(19) United States
(12) Patent Application Publication
(10) Pub. No.: US 2005/0123520 A1
(43) Pub. Date: Jun. 9, 2005

(54) Generation of living tissue in vivo using a mold

(76) Inventors: Roland D. Eavey, Brookline, MA (US); Syed H. Kamil, Shrewsbury, MA (US); Charles A. Vacanti, Lexington, MA (US); Martin P. Vacanti, Westborough, MA (US); Saim Aminuddin, Ampang Selangor (MY)

Correspondence Address: FISH & RICHARDSON PC, 225 FRANKLIN ST, BOSTON, MA, 02110, US

(21) Appl. No.: 10/838,758

(22) Filed: May 4, 2004

Related U.S. Application Data
(60) Provisional application No. 60/467,938, filed on May 5, 2003.

(51) Int. Cl. A61K 45/00

(52) U.S. Cl. 424/93.7

(57) ABSTRACT

The invention features methods of making living tissue constructs having a predetermined shape by obtaining a negative mold having a defined shape; suspending isolated tissue precursor cells in a hydrogel to form a liquid hydrogel-precursor cell composition; filling the liquid hydrogel-precursor cell composition into the mold; implanting the filled mold into a living host for incubation; removing the mold after a suitable incubation period; and removing the living tissue construct from the mold. The final living tissue construct can then be surgically implanted into a patient.

### CLAIM OF PRIORITY

This application claims priority under 35 USC § 119(e) to U.S. Provisional Patent Application Ser. No. 60/467,938, filed on May 5, 2003, the entire contents of which are hereby incorporated by reference.

### TECHNICAL FIELD

This invention relates to tissue engineering, materials science, cell biology, and plastic surgery.

### BACKGROUND

Over one million surgical procedures in the United States each year involve bone and cartilage replacement (Langer et al., 1993, Science, 920:260-266). The reconstruction of the anatomy of the head and neck presents a considerable challenge because of the unique geometries of facial structures, e.g., the ears and nose, which require a high degree of complexity and precision in implant fabrication. The use of allografts for these applications is limited by immunological complications, transmission of infectious diseases from the donor, premature resorption of the transplant, and lack of the ability and availability of donor material. Consequently, the use of autologous cartilage and/or bone grafts is considered a primary option. See, e.g., Lovice et al., 1999, Otolaryngol. Clin. N. Am., 32:113-139. However, tissues from locations such as the rib or iliac crest are limited in supply, are associated with significant donor site morbidity, and require significant surgical time to generate an appropriately shaped implant.

The use of pre-shaped prosthetic implants made from materials such as polyethylene, silicone, or polytetrafluoroethylene (PTFE) is common, but can be complicated due to higher infection rates and eventual protrusion of implants at the site of the procedure (Cohen et al., 1999, Facial Past. Surg. Clin. N. Am., 7:17-41).

Tissue engineering involves the regeneration of tissues such as bone and cartilage by seeding cells onto a customized biodegradable polymer scaffold to provide a three dimensional environment that promotes matrix production. This structure anchors cells and permits nutrition and gas exchange with the ultimate formation of new tissue in the shape of the polymer material. See, e.g., Vacanti et al., 1994, Transplant. Proc., 26:3309-3310; and Puelacher et al., 1994, Biomaterials, 15:774-778.

Specific disorders of the ear include congenital ear deformities known as microtia and atresia. In Grade I microtia, the ear is smaller than normal, but maintains most of the features of a normal ear. In Grade II microtia, the ear has an oblong elevation as well as a hook-form at the upper end. In Grade III, or “classic” microtia, the ear consists of a vertical skin appendage with a malformed lobule (earlobe) on the lower end. Most patients with the most severe form of microtia also lack an external auditory canal, also known as “atresia.” Given the present surgical techniques and materials, microtia surgical reconstruction remains a challenge.

### SUMMARY

The invention is based, in part, on the discovery that a detailed, well-defined, three-dimensional living tissue construct, such as a new ear or nose, can be formed by filling a mold with a hydrogel-precursor cell composition, and then implanting the filled mold into a person or animal for a time sufficient to grow the final living tissue construct.

In general, the invention features methods of making living tissue constructs having a specific, e.g., predetermined, shape by obtaining a negative mold having a predetermined, three-dimensional shape; suspending isolated tissue precursor cells in a hydrogel to form a liquid hydtogel-precursor cell composition; filling the liquid hydrogel-precursor cell composition into the mold; optionally inducing, e.g., controllably inducing, gel formation to solidify or partially solidify the liquid hydrogel-precursor cell composition; implanting the filled mold into a living host, e.g., a person or animal, to incubate the mold for a time sufficient to enable the cells to grow and form the tissue construct; and removing the living tissue construct from the mold. In some embodiments, the incubation is selected such that at least 50% (e.g., at least 75, 85, 90, 95, 98, or even 100%) of the hydrogel is degraded and removed from the mold (by normal physiological processes)

For example, the cells can be epidermal cells, chondrocytes and other cells that form cartilage, macrophages, adipocytes, dermal cells, muscle cells, hair follicles, fibroblasts, organ cells, osteoblasts, osteocytes and other cells that form bone, endothelial cells, mucosal cells, pleural cells, ear canal cells, tympanic membrane cells, peritoneal cells, Schwann cells, comeal epithelial cells, gingiva cells, central nervous system neural stem cells, or tracheal epithelial cells.

The hydrogels can be alginate (e.g., at a concentration of 0.5% to 8% or 1% to 4%, e.g., 2%), chitosan, pluronic, collagen, or agarose. The hydrogels can also be polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), or sulfonated polymers. In these methods and depending on the hydrogel, gel formation can be induced by contacting the liquid hydrogel with a suitable concentration of a divalent cation, such as Ca++, e.g., at a concentration of 0.2 g/ml of an alginate solution.

In another aspect, the invention features methods of reconstructing an anatomical feature in a mammal by obtaining a suitable negative mold having a three-dimensional negative shape of the anatomical feature; suspending isolated tissue precursor cells in a hydrogel to form a liquid hydrogel-precursor cell composition; filling the liquid hydrogel-precursor cell composition into the mold; optionally inducing gel formation to solidify the liquid hydrogel-precursor cell composition; implanting the filled mold into a host for a sufficient incubation period; removing the mold from the host; removing the tissue construct from the mold; and implanting the tissue construct into a recipient mammal, such as a person, dog, cat, horse, or other domesticated animal. Alternatively, the method can include obtaining a living tissue construct having the three-dimensional shape of the anatomical feature; and implanting the tissue construct into the mammal. In this method, the construct can be prepared by the new methods described herein.

The invention also features the injection-molded living tissue constructs made by the new methods. These constructs can have a variety of shapes, e.g., they can be in the shape of articular cartilage adjacent a joint, a bone, a portion of a bone, or a bone defect.

A “hydrogel” is a substance formed when an organic polymer (natural or synthetic) is set or solidified to create a three-dimensional open-lattice structure that can entrap molecules of water or other solution to form a gel. The solidification can occur, e.g., by aggregation, coagulation, hydrophobic interactions, or cross-linking. The hydrogels employed in this invention rapidly solidify to keep the cells evenly suspended within a mold until the gel solidifies. The hydrogels are also biocompatible, e.g., not toxic, to cells suspended in the hydrogel.

A “hydrogel-cell composition” or “hydrogel-precursor cell composition” is a suspension of a hydrogel containing desired tissue precursor cells. These cells can be isolated directly from a tissue source or can be obtained from a cell culture. A “tissue” is a collection or aggregation of particular cells embedded within its natural matrix, wherein the natural matrix is produced by the particular living cells. A “living tissue construct” is a collection of living cells that have a defined shape and structure. To be “living,” the cells must at least have a capacity for metabolism, but need not be able to grow or reproduce in all embodiments. Of course, a living tissue construct can also include, and in some embodiments preferably includes, cells that grow and/or reproduce. “Tissue precursor cells” or “precursor cells” are cells that form the basis of new tissue. Tissue cells can be “organ cells,” which include hepatocytes, islet cells, cells of intestinal origin, muscle cells, heart cells, cartilage cells, bone cells, kidney cells, cells of hair follicles, cells from the vitreous humor in the eyes, cells from the brain, and other cells acting primarily to synthesize and secret, or to metabolize materials. In some embodiments, these cells can be fully mature and differentiated cells. In addition, tissue precursor cells can be so-called “stem” cells or “progenitor” cells that are partially differentiated or undifferentiated precursor cells that can form a number of different types of specific cells under different ambient conditions, and that multiply and/or differentiate to form a new tissue.

An “isolated” tissue precursor cell, such as an isolated nerve cell, or an isolated nerve stem or progenitor cell or bone cell, or bone stem or progenitor cell, is a cell that has been removed from its natural environment in a tissue within an animal, and cultured in vitro, at least temporarily. The term covers single isolated cells, as well as cultures of “isolated” stem cells, that have been significantly enriched for the stem or progenitor cells with few or no differentiated cells.

As used herein, “negative mold” means a concave mold into which a liquid can be introduced for subsequent solidification. The mold is “negative” in the sense that concavity of the mold represents convexity in the object to be formed.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, useful methods and materials are described below. All publications, patent applications, patents, and other references mentioned herein are incorporated by reference in their entirety. In case of conflicting subject matter, the present specification, including definitions, will control. In addition, the materials, methods, and examples are illustrative only and not intended to be limiting.

The invention has many advantages. For example, the new methods reduce the number of manufacturing steps needed to prepare precise, three-dimensional biological tissues. The new methods also provide increased uniformity of cell seeding throughout the construct, and increased efficiency of cell containment within the construct.

Other features and advantages of the invention will be apparent from the following detailed description, and from the claims.

### BRIEF DESCRIPTION OF THE DRAWINGS

FIG. 1 is a schematic diagram of the molding process. Cartilage is harvested and digested. Chondrocytes are concentrated and suspended in a hydrogel solution, e.g., 2% alginate. Immediately before injection into the mold, sterilized CaSO₄ is mixed with chondrocytes in alginate to initiate gel formation. The chondrocyte/alginate/CaSO₄ mixture is injected into a sterilized mold using a syringe and needle.

FIG. 2 is a representation of two halves of a mold made of gold and shaped like a human auricle with perforating holes in both halves of the mold.

FIG. 3A is a representation of a human sized and shaped auricle within the mold after growth in a pig after eight weeks.

FIG. 3B is a representation of the human sized and shaped auricle taken out of the mold.

FIG. 4 is a representation of a human sized and shape auricle grown inside a mold in a sheep after twelve weeks.

FIGS. 5A and 5B are representations of micrographs of tissue-engineered cartilage grown in calcium alginate hydrogel (FIG. 5A) and Pluronic F-127® hydrogel (FIG. 5B), with Safranine-O staining at a magnification of 100×.

### DETAILED DESCRIPTION

The invention provides improved tissue engineering techniques and improved living tissue constructs or implants. In contrast to conventional tissue engineering techniques, that involve creating a shaped scaffold, and then seeding the shaped scaffold with cells in a separate step, the invention utilizes a suspension of cells in a solution from which a hydrogel is formed in the shape of the desired tissue construct within a 3-D mold. The mold is then implanted into a person or animal to be incubated for a time sufficient to allow the cells to grow and form the final living tissue construct.

The new methods can be used to grow new tissue such as, for example, cartilage, bone, skin, epithelial layers, new organs, and central nervous system tissue, by using one or more hydrogel-cell compositions that are formed into a precise shape using new molding techniques. To guide the development and shape of the new tissue, a precise negative mold is created from an inert material (e.g., gold, titanium, platinum, or inert biocompatible plastics), and the hydrogel-cell composition is delivered into the mold and, optionally, cured or set, or partially cured or set, to form a solid or semi-solid, three-dimensional living tissue construct. The filled mold is then implanted into a living host (a person or animal) for a time sufficient to allow the cells to grow and form the final tissue construct, and to allow the hydrogel to degrade and be removed from the mold by normal biological processes in the host. Thus, the weight of the final tissue construct is comprised largely of cells, with a low percentage, e.g., no more than 0, 1, 2, 5, 10, 15, 20, 25, 30, 40, or 50 percent by weight of the hydrogel, depending on the nature of the hydrogel. For example, a highly non-immunogenic hydrogel can be present in the final tissue construct at somewhat higher concentrations than a hydrogel that poses some risk of causing an immune reaction over time. However, under ideal conditions, the final tissue construct would include no hydrogel, and be composed entirely of cells (from the original tissue precursor cells as well as grown into the construct from the surrounding bodily tissues, e.g., blood vessels).

In the following subsections, suitable molding techniques, hydrogels, cells, and delivery methods will be described, along with illustrative examples.

General Methodology

As with any process based on molding, e.g., injection molding, the size and shape of the shaped living tissue construct is determined by the size and shape of the negative mold. Thus, the invention can be employed to produce a living, biological tissue implant or construct having essentially any size and shape, with the size and shape being precisely controlled. The living tissue construct can be used for the repair, reconstruction, or modification of external or internal anatomical structures. In some embodiments, the construct is a precisely shaped piece of cartilage for the reconstruction of an external anatomical structure, e.g., an ear or a nose. In other embodiments, the tissue construct is a precisely shaped piece of cartilage for the reconstruction of an internal anatomical structure such as a meniscus in the knee. In yet other embodiments, the biological implant is a precisely shaped piece of bone for the repair of a skeletal defect or injury. For example, pieces of bone can be produced for reconstruction of facial bones, following severe facial injuries in an automobile accident.

Because injection molding allows for the use of a precise negative mold, detailed anatomical information from MRI or CT devices can be utilized to maximum advantage. For example, data output from an MRI or CT device can serve as input for computer aided drafting/computer aided manufacturing (CAD/CAM) and rapid prototyping to produce high quality molds in which the biological tissue constructs are formed. CAD/CAM hardware and software are commercially available and can be employed using techniques known in the art, to design and produce molds suitable for use in the invention.

The principle of using MRI and CT data to fabricate custom-designed implants has been demonstrated using molded silicone (See, e.g., Binder and Kaye, 1994, Plast. Recon. Surg., 94:775-785). A similar procedure can be utilized as described here to produce custom-designed implants from living tissues such as cartilage or bone.

Although CAD/CAM techniques can be used in the design and production of molds they are not required. In some embodiments of the invention, a mold is constructed manually, e.g., by casting a gold (or other inert metal) mold using standard casting techniques, or by using a Silastic ERTV mold making kit (Dow Corning)(other inert plastics can be used as well). For example, negative molds can be fabricated by immersing half of a positive model in a bed formed from the mixed components of an ERTV kit. This mixture is then placed in an 80° F. oven for 30 minutes. After the bottom is hardened, approximately the same amount of uncured silastic is poured on top to a height of 2 cm. This is again cured at 80° F. for 30 minutes. After separation of the top and lower sets of the mold, the model is removed.

In general, the molds can be made of any materials that can be filled with a hydrogel and maintain their shape over time when implanted into a person or animal, and do not cause inflammation or a foreign body reaction in immuno-competent animals or people.

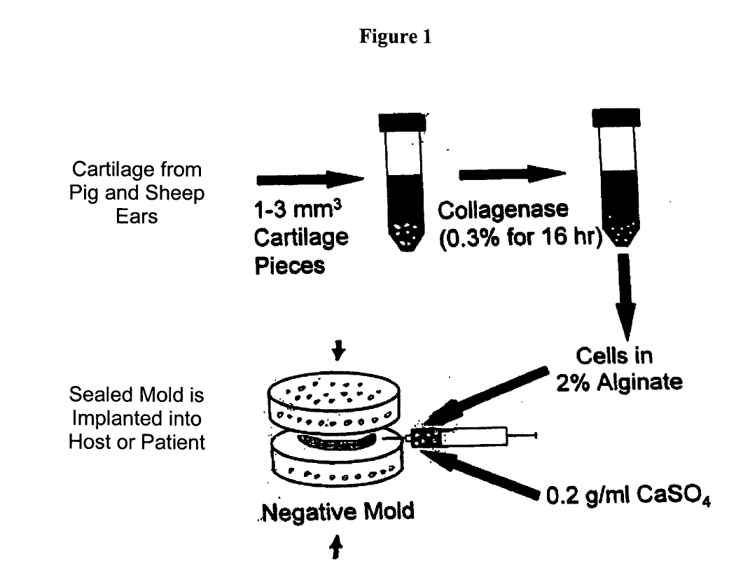

As shown in FIG. 1, cells are extracted from a source, such as a bone or cartilage, using standard techniques. For example, cartilage can be cut into small pieces of 1 to 3 mm³, and then disrupted with an enzyme or other chemical that separates the cells but does not destroy them. For example, collagenase works well for disrupting collagen into separate cells. The cells are then suspended in a hydrogel, such as 2% alginate, to produce a hydrogel-cell composition that can be delivered in liquid form, and is then filled, e.g., injection molded, into a pre-constructed negative mold. The hydrogel-cell composition can be introduced into the mold simultaneously with a curing composition, such as CaSO₄ in the case of alginate. After a predetermined time to allow the hydrogel to partially or completely cure or set, such as 15 minutes for alginate, the filled mold is either implanted directly into a living host (e.g., a person or animal). The host can be the same or different from the recipient who eventually receives the tissue construct. Alternatively, the filled mold is temporarily put into a culture medium for a time sufficient to allow the cells to grow (or at least until a surgical site for implantation into the host is prepared), and is then implanted into the host.

The filled mold is left inside the host person or animal for a time sufficient for the cells to grow and fill the mold, while the hydrogel slowly biodegrades. For alginate and cartilage, the sufficient time is between about 10 and 20 weeks. To grow bone, the sufficient time will be longer, e.g., about 15 to 40 weeks. To allow for nutrients to flow into the filled mold, and waste products secreted by the cells to exit the mold, the entire mold is provided with small perforations (e.g., 10μ to 0.9 mm). The size and locations of these perforations on the mold are adjusted depending on the size of the tissue construct, the diffusivity of the hydrogel, and the type of cells. In some embodiments, the perforations are made to be somewhat smaller than the size of the cells to be used (e.g., if the hydrogel is of a small molecular weight and tends to leak out of the mold). For example, chondrocytes are about 15 to 29μ in diameter. Thus, the perforations can be made to have a diameter of about 12 to 15μ to prevent the cells from escaping the mold. Thus, the hydrogel need not be selected for its ability to keep cell in the mold. In some embodiments, it is useful to have at least some of the holes with a size that permits blood vessels to grow through the holes and into the mold. The perforations can be made using drills, needles, or lasers, and can be made after the mold if formed, or during generation of the mold.

In certain embodiments, the filled mold is implanted into a host animal or person other than the recipient, i.e., the person or animal who will receive the final tissue construct. For example, if the recipient is man A, the host can be man B or an animal. In those situations, the host's immune system may react against the cells within the mold if the cells are taken from the eventual recipient (no such problems arise if the cells are harvested from the host). To avoid this reaction, the host's immune system can be down-regulated (immunosuppressed), e.g., with known drugs such as steroids and other immunosuppressant drugs such as cyclosporine, or the host can be selected for its lack of an active immune system. Alternatively, the hydrogel can be selected to have openings or pores in its open-lattice polymer structure of the proper size to allow cellular nutrients to pass through, but to exclude immune system components (such as antibodies). This concept is discussed in a bit more detail below.

In other embodiments, the cells can be harvested from the host, and the filled mold can be incubated in the host. In this scenario, if the recipient is different from the host, the recipient's immune system may need to be suppressed. Therefore, the cells are typically harvested from the recipient, and the filled mold is incubated in the recipient to avoid (as much as possible) immune reactions to the cells.

Hydrogels

Any suitable polymer hydrogel can be used in methods of the invention. A suitable polymer hydrogel is one that is biologically compatible, non-cytotoxic, and formed through controllable crosslinking (gelation), under conditions compatible with viability of isolated cells suspended in the solution that undergoes gelation. Various polymer hydrogels meeting these requirements are known in the art and can be used in the practice of the invention. Examples of different hydrogels suitable for practicing this invention, include, but are not limited to: (1) hydrogels cross-linked by ions, e.g., sodium alginate; (2) temperature dependent hydrogels that solidify or set at body temperature, e.g., PLURONICS™; (3) hydrogels set by exposure to either visible or ultraviolet light, e.g., polyethylene glycol polylactic acid copolymers with acrylate end groups; and (4) hydrogels that are set or solidified upon a change in pH, e.g., TETRONICS™.

Examples of materials that can be used to form these different hydrogels include polysaccharides such as alginate, polyphosphazenes, and polyacrylates, which are cross-linked ionically, or block copolymers such as PLURONICS™ (also known as POLOXAMERS™), which are poly(oxyethylene)-poly(oxypropylene) block polymers solidified by changes in temperature, or TETRONICS™ (also known as POLOXAMINES™), which are poly(oxyethylene)-poly(oxypropylene) block polymers of ethylene diamine solidified by changes in pH.

Ionic Hydrogels

Ionic polysaccharides, such as alginates and chitosan, can be used to suspend living cells. Tissue precursor cells are mixed with a polysaccharide solution, the solution is delivered into a mold, and then solidifies when the proper concentrations of ions are added. For example, alginate is an anionic polysaccharide capable of reversible gelation in the presence of an effective concentration of a divalent cation. A hydrogel can be produced by cross-linking the anionic salt of alginic acid, a carbohydrate polymer isolated from seaweed, with ions, such as calcium cations. The strength of the hydrogel increases with either increasing concentrations of calcium ions or alginate. For example, U.S. Pat. No. 4,352,883 describes the ionic cross-linking of alginate with divalent cations, in water, at room temperature, to form a hydrogel matrix.

In a more specific example, Ca⁺⁺ can be supplied conveniently in the form of CaSO₄. In some embodiments of the invention, CaSO₄ is added in the amount of 0.1 to 0.5 gram, e.g., approximately 0.2 gram, per milliliter of a 2% solution of alginate. If the amount of soluble alginate is increased or decreased, the amount of divalent cation may need to be adjusted accordingly. Such adjustment is within ordinary skill in the art. The solubility of CaSO₄ is 0.209 g/ml, which is much lower than that of CaCl₂ (74.5 g/ml), which is the crosslinking agent typically used in for encapsulation of cells in alginate. See Beekman et al., 1997, Exper. Cell Res., 237:135-141. At a concentration of CaSO₄ near or above the solubility limit, Ca²⁺ in solution begins to crosslink alginate, and it is replenished by solubilization of precipitated CaSO₄. This results in a significant slowing of the crosslinking process. Such slowing can be advantageous, because it allows the alginate/CaSO₄ mixture to be injected into a mold before the completion of the crosslinking process occurs in the shaped implant.

In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole).

Polyphosphazenes are polymers with backbones consisting of nitrogen and phosphorous atoms separated by alternating single and double bonds. Each phosphorous atom is covalently bonded to two side chains. Polyphosphazenes that can be used have a majority of side chains that are acidic and capable of forming salt bridges with di- or trivalent cations. Examples of acidic side chains are carboxylic acid groups and sulfonic acid groups. Bioerodible polyphosphazenes have at least two differing types of side chains, acidic side groups capable of forming salt bridges with multivalent cations, and side groups that hydrolyze under in vivo conditions, e.g., imidazole groups, amino acid esters, glycerol, and glucosyl.

Bioerodible or biodegradable polymers, i.e., polymers that dissolve or degrade within a period that is acceptable in the desired application (in vivo “culturing” of the mold), will degrade in less than about 6 months and preferably in less than about 2 to 3 months, once exposed to a physiological solution of pH 6-8 having a temperature of between about 25° C. and 38° C. In some hydrogels, hydrolysis of a side chain results in erosion of the polymer. Examples of hydrolyzing side chains are unsubstituted and substituted imidizoles and amino acid esters in which the side chain is bonded to the phosphorous atom through an amino linkage.

Methods for synthesis and the analysis of various types of polyphosphazenes are described in U.S. Pat. Nos. 4,440,921, 4,495,174, and 4,880,622. Methods for the synthesis of the other polymers described above are known to those skilled in the art. See, for example Concise Encyclopedia of Polymer Science and Engineering, J. I. Kroschwitz, editor (John Wiley and Sons, New York, N.Y., 1990). Many polymers, such as poly(acrylic acid), alginates, and PLURONICS™, are commercially available.

Water soluble polymers with charged side groups are cross-linked by reacting the polymer with an aqueous solution containing multivalent ions of the opposite charge, either multivalent cations if the polymer has acidic side groups, or multivalent anions if the polymer has basic side groups. Cations for cross-linking the polymers with acidic side groups to form a hydrogel include divalent and trivalent cations such as copper, calcium, aluminum, magnesium, and strontium. Aqueous solutions of the salts of these cations are added to the polymers to form soft, highly swollen hydrogels.

Anions for cross-linking the polymers to form a hydrogel include divalent and trivalent anions such as low molecular weight dicarboxylate ions, terepthalate ions, sulfate ions, and carbonate ions. Aqueous solutions of the salts of these anions are added to the polymers to form soft, highly swollen hydrogels, as described with respect to cations.

For purposes of preventing the passage of antibodies into the hydrogel, but allowing the entry of nutrients, a useful polymer size in the hydrogel is in the range of between 10,000 D and 18,500 D. Smaller polymers result in gels of higher density with smaller pores.

Temperature-Dependent Hydrogels

Temperature-dependent, or thermosensitive, hydrogels can be use in the methods of the invention. These hydrogels have so-called “reverse gelation” properties, i.e., they are liquids at or below room temperature, and gel when warmed to higher temperatures, e.g., at or above body temperature. Thus, these hydrogels can be easily injected into a mold at or below room temperature as a liquid and automatically form a semi-solid gel when warmed to or above body temperature. Examples of such temperature-dependent hydrogels are PLURONICS™ (BASF-Wyandotte), such as polyoxyethylene-polyoxypropylene F-127, F-108, and F-68, poly (N-isopropylacrylamide), and N-isopropylacrylamide copolymers.

These copolymers can be manipulated by standard techniques to affect their physical properties such as porosity, rate of degradation, transition temperature, and degree of rigidity. For example, the addition of low molecular weight saccharides in the presence and absence of salts affects the lower critical solution temperature (LCST) of typical thermosensitive polymers. In addition, when these gels are prepared at concentrations ranging between 5 and 25% (W/V) by dispersion at 4° C., the viscosity and the gel-sol transition temperature are affected, the gel-sol transition temperature being inversely related to the concentration. These gels have diffusion characteristics capable of allowing cells to survive and be nourished.

U.S. Pat. No. 4,188,373 describes using PLURONIC™ polyols in aqueous compositions to provide thermal gelling aqueous systems. U.S. Pat. Nos. 4,474,751, ′752, ′753, and 4,478,822 describe drug delivery systems which utilize thermosetting polyoxyalkylene gels; with these systems, both the gel transition temperature and/or the rigidity of the gel can be modified by adjustment of the pH and/or the ionic strength, as well as by the concentration of the polymer.

pH-Dependent Hydrogels

Other hydrogels suitable for use in the methods of the invention are pH-dependent. These hydrogels are liquids at, below, or above specific pH values, and gel when exposed to specific pHs, e.g., 7.35 to 7.45, the normal pH range of extracellular fluids within the human body. Thus, these hydrogels can be easily delivered into a mold as a liquid and form a semi-solid gel when exposed to the proper pH. Examples of such pH-dependent hydrogels are TETRONICS™ (BASF-Wyandotte) polyoxyethylene-polyoxypropylene polymers of ethylene diamine, poly(diethyl aminoethyl methacrylate-g-ethylene glycol), and poly(2-hydroxymethyl methacrylate). These copolymers can be manipulated by standard techniques to affect their physical properties.

An example of another a useful pH-dependent hydrogel is collagen. Collagen is a protein that undergoes cross-linking in response to shift in pH from alkaline to acid, e.g., a shift from a pH in the range of <2 to a pH in the range of >6. See, e.g., Bell et al., 1979, Proc. Nat. Acad. Sci., 76:1274.

Light Solidified Hydrogels

Other hydrogels that can be used in the methods of the invention are solidified by either visible or ultraviolet light. These hydrogels are made of macromers including a water-soluble region, a biodegradable region, and at least two polymerizable regions as described in U.S. Pat. No. 5,410,016. For example, the hydrogel can begin with a biodegradable, polymerizable macromer including a core, an extension on each end of the core, and an end cap on each extension. The core is a hydrophilic polymer, the extensions are biodegradable polymers, and the end caps are oligomers capable of cross-linking the macromers upon exposure to visible or ultraviolet light, e.g., long wavelength ultraviolet light. These types of hydrogels can be used with transparent or translucent molds, or with molds that have optic fibers that introduce light into the mold.

Examples of such light solidified hydrogels include polyethylene oxide block copolymers, polyethylene glycol polylactic acid copolymers with acrylate end groups, and 10K polyethylene glycol-glycolide copolymer capped by an acrylate at both ends. As with the PLURONIC™ hydrogels, the copolymers comprising these hydrogels can be manipulated by standard techniques to modify their physical properties such as rate of degradation, differences in crystallinity, and degree of rigidity.

Tissue Precursor Cells

Various types of isolated cells or tissue precursor cells (e.g., progenitor or stem cells) can be used in methods according to the invention. Selection of cell type will depend on the type of construct to be produced. For example, isolated chondrocytes are used for production of a shaped cartilage tissue construct such as an ear or nose. Isolated osteocytes are used for production of shaped bone constructs. Isolated adipocytes are used for production of shaped adipose tissue constructs. Myoblasts are used for production of a shaped muscle tissue constructs.

Tissue precursor cells can be obtained directly from a mammalian donor, e.g., a patient's (recipient's) own cells, from a culture of cells from a donor, or from established cell culture lines. For example, the mammal can be a mouse, rat, rabbit, guinea pig, hamster, cow, pig, horse, goat, sheep, dog, cat, and of course, the donor can be a human, e.g., the host or the recipient (patient). Cells of the same species and preferably of the same immunological profile can be obtained by biopsy, either from the recipient/patient or a close relative of the recipient of the living tissue construct. Using standard cell culture techniques and conditions, the cells are then grown in culture until confluent and used when needed. The cells are preferably cultured only until a sufficient number of cells have been obtained for a particular application.

If cells are used that may elicit an immune reaction, such as human muscle cells from an immunologically distinct donor, then the recipient can be immunosuppressed as needed, for example, using a schedule of steroids and other immunosuppressant drugs such as cyclosporine. However, the use of autologous cells will avoid such an immnunologic reaction.

Cells can be obtained directly from a donor, washed, suspended in a selected hydrogel before being injected into a mold. To enhance cell growth, the cells are added or mixed with the hydrogel just prior to injection into the mold. Cells obtained by biopsy are harvested, cultured, and then passaged as necessary to remove contaminating, unwanted cells. The isolation of chondrocytes is described in the examples below. Cell viability can be assessed using standard techniques including visual observation with a light or scanning electron microscope, histology, or quantitative assessment with radioisotopes. The biological function or metabolism of the cells can be determined using a combination of the above techniques and standard functional assays.

Examples of cells that can be delivered into molds and subsequently grow new tissue in living tissue constructs include epidermal cells; chondrocytes and other cells that form cartilage (“cartilage-forming cells”); macrophages; dermal cells; muscle cells; hair follicles; fibroblasts; organ cells; osteoblasts, periosteal cells, and other cells that form bone (“bone forming cells”); endothelial cells; mucosal cells, e.g., nasal, gastric, bladder and oral mucosal cells; pleural cells; ear canal cells; tympanic membrane cells; peritoneal cells; Schwann cells; corneal epithelial cells; gingiva cells; tracheal epithelial cells; and neural cells, including neuronal stem cells and neurons.

In some embodiments, two or more different cell types are used to prepare one tissue construct. For example, to make a “skin” tissue construct, one could take dermal cells to make one layer, and epidermal cells to make a second layer. The skin mold would be in the shape of a defect or wound in the skin that is to be repaired.

To make an ear, one can coat the inside walls of an ear-shaped mold with a first hydrogel-cell composition that contains fibroblasts and/or other skin cells to form a thin layer of a “skin” surrogate (or use two layers or coatings, with an epidermal cell layer closest to the mold wall, and an inner coating of a dermal cell layer), and then fill the remaining void in the coated mold with a second hydrogel-cell composition containing cartilage forming cells. The first hydrogel-cell composition can be sprayed or otherwise coated onto the inner walls of the mold, or the mold can be dipped in the first hydrogel-cell composition. Alternatively, the mold can be filled with this first hydrogel-cell composition, and allowed to cure or set only immediately adjacent to the walls only, e.g., by temperature, or by having a curing agent coated on the inner wall surfaces. The remaining uncured first hydrogel-cell composition is then poured out of the mold, and the second hydrogel-cell composition is filled into the pre-coated mold and set or cured. The mold filled with the two different hydrogel-cell compositions is then implanted into a host.

Preparation of Hydrogel-Cell Compositions

First, a hydrogel of choice is prepared using standard techniques. For example, a biodegradable, thermosensitive polymer at a concentration ranging between 5 and 25% (W/V) is useful for the present invention. If the hydrogel is an alginate, it can be dissolved in an aqueous solution, for example, a 0.1 M potassium phosphate solution, at physiological pH, to a concentration between about 0.1 to about 4% by weight, e.g., 2%, to form an ionic hydrogel.

Second, isolated tissue precursor cells are suspended in the polymer solution at a concentration mimicking that of the tissue to be generated. The optimal concentration of cells to be delivered into the mold is determined on a case by case basis, and may vary depending on cellular type and the region of the patient's body into which the living tissue implant is inserted. Optimization experiments require modifying only a few parameters, i.e., the cell concentration or the hydrogel concentration, to provide optimal viscosity and cell number to support the growth of new tissue. For chondrocytes, the cell concentration range is from about 10 million cells/ml to about 100 million cells/ml, e.g., from about 25 million cells/ml to about 50 million cells/ml, e.g., 40-50 cells/ml.

Implantation of Filled Molds into a Host

The filled molds are then implanted, e.g., subcutaneously (into a so-called “skin pocket”), intramuscularly, or intraperitoneally, into a host. For example, a human tissue construct can be implanted into a pig, dog, or primate host, but typically, the host will be the recipient of the tissue construct or a close relative.

The filled mold is implanted surgically, and the mold is held in place, e.g., with sutures or tissue anchors, e.g., screw, clamps, or staples. The wound is then closed, because the mold will remain within the host for a period of several weeks to several months. Once the tissue construct in complete, i.e., the cells have fully grown throughout the construct, and the hydrogel has degraded and been removed from the mold, the mold is surgically removed, and opened to remove the final tissue construct. The incubation period within the host ranges from a minimum of about 6 to 10 weeks, e.g., 8 or 9 weeks, to a maximum of about 4 to 6 months or even longer, e.g., 2, 3, 4, 5, 6, 7, or 8 months. The time sufficient for the incubation period depends on the nature of the cells, the hydrogel, the mold, and the size of the tissue construct. The larger the construct and the denser the tissue construct, the longer the incubation period. For example, to prepare a cartilage ear using an alginate hydrogel, the incubation period should be between about 10 and 20 weeks. Note that 12 weeks is said to be the general degradation time for alginate in the subcutaneous space (See, Suzkuki et al., 1999, J. Biomed. Mat. Res., 48:522-527), but this time depends on the nature and concentration of the alginate.

Implantation of Living Tissue Constructs into a Recipient

To implant a living tissue construct into a human or animal recipient, the implantation site of the patient can be exposed by surgical resection and the tissue construct implanted directly at that site. Alternatively, if the construct is small enough, the implantation site can be viewed with the aid of, e.g., an endoscope, laparoscope, arthroscope, or esophagoscope, all of which can be modified to include a mechanical articulation and delivery system for implanting the tissue construct through a small incision. During implantation, the site is cleared of bodily fluids including blood, e.g., with a burst of air or suction. Thus, the hydrogel-cell-containing tissue construct can be introduced through a laparoscope, endoscope, laryngoscope, cystoscope, proctoscope, or thoracoscope to any the interior surface of any lumen or cavity, or other surfaces, such as intraperitoneal, extraperitoneal, and thoracic cavity, and then implanted into the desired space.

The final tissue construct is removed from the mold, and cleared of any debris, e.g., by rinsing with sterile saline or other physiological fluid. The construct can be implanted directly after removal from the mold, or can be temporarily placed into an in vitro culture vessel containing human plasma or blood, e.g., from the recipient, and maintained at body temperature, until the implantation site is prepared.

Throughout the implantation procedure, the amount of trauma caused to the cells during the delivery and implantation steps can be determined by measuring a biological function specific for the cells being used. For example, when chondrocytes are being applied, the integrity of the new cartilage can be evaluated by standard biomechanical stress analyses, such as determination of compression moduli.

Applications

Since the hydrogel-cell compositions can support many different kinds of tissue precursor cells and the molding methods can be used to create virtually any three-dimensional shape, the new methods can be used in any instance in which it desirable to generate new tissue. Some of the applications are to treat cartilage pathologies in a variety of tissues, e.g., facial-plastic surgery (auricular and nasal pathologies), repair of cartilaginous defects in the articular surfaces of various joints (degenerative diseases and trauma), and laryngeotracheal reconstruction (subglottic stenosis and tracheal resection for cancer). For example, the mold can be designed in the shape of a portion of the trachea to treat tracheal stenosis. Tissue engineered bone, muscle, tendon, cardiovascular system structures (heart valves and blood vessels), trachea, and other structures can also be generated in desired shapes and sizes utilizing the new methods. The new methods therefore can be useful in different types of tissue regeneration in multiple locations and organs of the body.

Particular applications described below relate to the generation of cartilage, bone, and neural tissues.

Treatment of Cartilage Defects

Cartilage is a specialized type of dense connective tissue consisting of cells embedded in a matrix. There are several kinds of cartilage. Hyaline cartilage is a bluish-white, glassy translucent cartilage having a homogeneous matrix containing collagenous fibers that is found in articular cartilage, in costal cartilages, in the septum of the nose, and in the larynx and trachea. Articular cartilage is hyaline cartilage covering the articular surfaces of bones. Costal cartilage connects the true ribs and the sternum. Fibrous cartilage contains collagen fibers. Yellow cartilage is a network of elastic fibers holding cartilage cells which is found primarily in the epiglottis, the external ear, and the auditory tube. By harvesting the appropriate chondrocyte precursor cells, any of these types of cartilage tissue can be grown using the methods of the invention.

For example, new tissue can be grown for a cartilage meniscus replacement in the knee. A negative mold is prepared to provide a tissue construct in the shape of the meniscus to be replaced. Thereafter, a liquid hydrogel-chondrocyte composition is injected into the mold. The hydrogel subsequently solidifies, taking the shape of the desired meniscus replacement and providing a suspension for the chondrocytes that permits diffusion of nutrients and waste products to and from the suspended chondrocytes. After incubation in a host for at least 15 weeks, final new tissue construct is removed from the mold, and is implanted into the knee using the standard surgical techniques. Over time, e.g., over a period of approximately six weeks, the construct will become vascularized and the chondrocytes will grow to engraft to existing tissue.

Treatment of Microtia

The human ear has a highly developed and intricate 3-D topography. This shape can be uniquely replicated using the new methods. Using this method, a concentration of autologbus chondrocytes is delivered into a mold in combination with a polymer hydrogel. The mold is perforated to enable nutrition of the chondrocytes. The mold filled with the hydrogel-cell composition is implanted subcutaneously into a host, and retrieved after a suitable incubation period. The cartilage generated inside the mold is removed and implanted into a patient to treat microtia.

Treatment of Bone Defects

In another example, periosteal cells (i.e., bone-growing cells) can be used in the invention to fill bone defects or to prepare entire new bones. First, a negative mold is prepared to fit the dimensions of the bone defect (e.g., by creating a positive model of the bone defect with a plastic material that is filled into the defect while in paste or gel form and then solidifies). The negative mold is prepared from the plastic positive model. The hydrogel-periosteal-cell composition can then be delivered into the mold. Once again the hydrogel solidifies, i.e., suspends and maintains the cells. After the tissue construct is solidified, it is implanted into a host for incubation. Then the mold is removed from the host, opened, and the final bone tissue construct is removed from the mold and implanted into the bone defect and subsequently grows to fill in the bone defect.

In order that the invention may be more fully understood, the following examples are provided. The examples are for illustrative purposes only, and they are not to be construed as limiting the scope or content of the invention in any way.

### EXAMPLES

Experiments were conducted to develop methods to create a human ear, which is a structure of complex geometry. A mold made of perforated gold was used and various chondrocyte/polymer compositions were filled into the mold. After subcutaneous implantation of the filled mold into an animal host for incubation, the final tissue constructs were removed and had developed morphology that closely resembled that of native cartilage in the form of a human ear.

### Example 1

### Preparation of a 3-D Ear-Shaped Mold

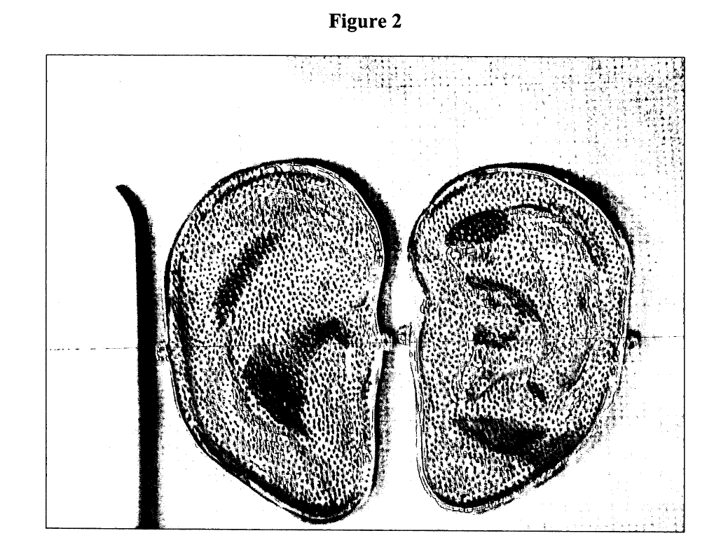

Pure gold (24 carat) was used to make the mold in the shape of complete auricle. The mold was made by first making a wax impression of a human ear, and then using a standard “lost-wax” process to create the final gold mold. The mold (FIG. 2) was designed in two separate halves, which were united with small gold screws. The two halves were hollow and when joined created the auricular shape internally. Numerous perforating holes of 0.5-0.7 mm diameter were drilled on both halves of the mold, covering the surface.

### Example 2

### Isolation of Chondrocytes

Auricular cartilage was harvested from a total of 7 pigs and 3 sheep under general anesthesia. Perichondrium was removed under sterile conditions and the cartilage was fragmented into small pieces; washed in phosphate-buffered saline (PBS) solution containing 100 μL of penicillin, 100 mg/L of streptomycin and 0.25 mg/L of amphoterecin B (Gibco, Grand Island, N.Y.); and digested with 0.3% collagenase II (Worthington Biochemical Corp., Freehold, N.J., USA) for 8-12 hours. The resulting cell suspension was passed through a sterile 250-micron polypropylene mesh filter (Spectra/Mesh 146-426; Spectrum Medical Industries, Inc., Laguna Hills, Calif.). The filtrate was centrifuged at 6000 rpm, and the resulting cell pellet was washed twice with copious amount of Dulbecco's phosphate buffered-saline (PBS) (Gibco, Grand Island, N.Y., USA) without Ca²⁺. Cell number was determined using a hemocytometer and the cell viability determined using trypan blue dye (Sigma-Aldrich, Irvine, Kans., USA.).

### Example 3

### Hydrogel-Cell Compositions

The chondrocyte suspensions demonstrating cell viability in excess of 85% were suspended in each of the three biocompatible polymers (calcium alginate, Pluronic F-127® and polyglycolic acid) at a concentration of 50 million cells/cc. A total of 10 molds were implanted: 3 contained a mixture of Pluronic F-127 and chondrocytes; a further 3 contained calcium alginate with chondrocytes; and another 3 combined polyglycolic acid (PGA) fibers and attached chondrocytes. One mold was used as a control without any polymer or cells inside.

Alginate was prepared by dissolving ultra pure sodium alginate (0.1 M K₂HPO₄, 0.135 M NaCl, pH 7.4) (Pronova, Portsmouth, N.H.) in PBS and was filtered though a 0.22 micron filter. A total of 5 ml of alginate was used to add to the cells after the formation of a pellet by centrifuging the cell suspension to achieve a concentration of 40-50 million cells/ml. Polymerization of the alginate was achieved prior to injection of the mixture inside the mold by the addition of the curing agent CaSO₄ (0.04 ml/ml of alginate), sterilized previously by autoclave. Once the mixture of alginate and chondrocytes (and curing agent) was transferred inside the mold using a 10 ml syringe, the mold was immersed in the solution of CaCl for ten seconds before its implantation.

FIG. 1 illustrates the overall method of isolating the cartilage, mixing the cells with alginate and the curing agent, and injecting the hydrogel-cell composition into a mold.

In a similar manner, two other hydrogels were used to prepare hydrogel-chondrocyte compositions. Pluronic F-127® consists by weight of approximately 70% ethylene oxide and 30% propylene oxide. This material is soluble in water and becomes a hydrogel at room temperature. An aliquot of chondrocyte suspension was mixed at 4° C. with a 30% solution of Pluronic F-127 at a cellular concentration of 50 million cell/ml. A total of 5 ml of the polymer was, used. At room temperature, this mixture of chondrocytes and Pluronic F-127 became gel-like in consistency and was transferred by syringe to completely fill the interior of the mold.

Sheets of polyglycolic acid fibers of 100 μm thickness (PGA, Davis & Geck, Danbury, Conn.) were used for the seeding of the chondrocytes. Suspensions were created by mixing the cells with Ham F-12 culture medium (Life technologies, Baltimore, Md.) to a cellular density of 50 million cells/ml. The chondrocytes were seeded onto the PGA. The PGA fibers and chondrocytes were placed in a Petri dish for 5 days in Ham F-12 culture with L-glutamine, 50 mg/L L-ascorbic acid, 100 μ/L of penicillin, 100 mg/L of streptomycin, 0.25 mg/L of amphoterecin B, supplemented with 10% fetal bovine serum (Sigma-Aldrich, St. Louis, Mo.). The cell cultures were maintained at 37° C. and 5% CO₂. The culture media was changed twice daily. Once the cells attached to the PGA fibers (as observed by microscopy), the material was transferred inside the mold.

### Example 4

### In Vivo Implantation into a Host

Under general anesthesia, the ventral surface of the pig or sheep was cleaned and draped. A 6 to 7 cm linear skin incision was made and a subcutaneous skin pocket was created. The gold mold with the mixture of polymer and chondrocytes was placed inside the skin pocket. Hemostasis was achieved before closing the wound with 3-0 polyglactin absorbable sutures.

### Example 5

### Analysis of Cartilage Tissue Constructs

The development of cartilage in vivo in the constructs within the molds after implantation into the hosts was analyzed after the molds were removed. The specimens were harvested after 8 to 20 weeks. Samples were obtained for histological analysis and were fixed in 10% phosphate-buffered formalin (Fisher Scientific, Fair Lawn, N.J.). Once fixed for at least 24 hours, specimens were embedded in paraffin and sectioned using standard histochemical techniques. Slide sections were stained with hematoxylin & eosin, and Safranine-O stains.

Gross Morphology

The constructs were removed carefully and examined for gross morphological features of external size, shape, structural details and texture of the tissue to palpation. All the molds except the control were able to generate tissue in the desired shape of an auricle. The constructs obtained from calcium alginate demonstrated the most natural appearance regarding external shape and anatomical details plus tactile texture

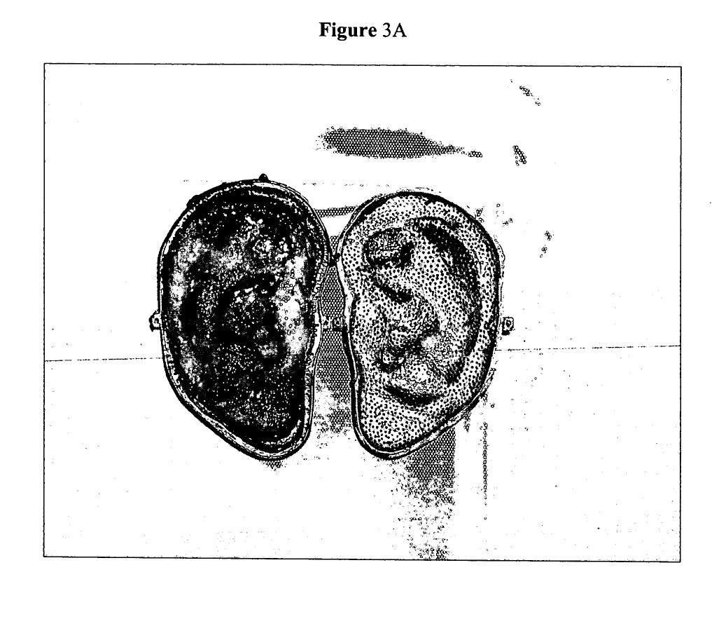

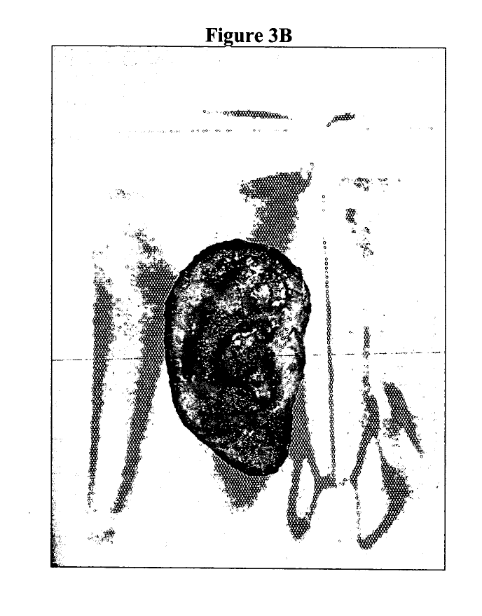

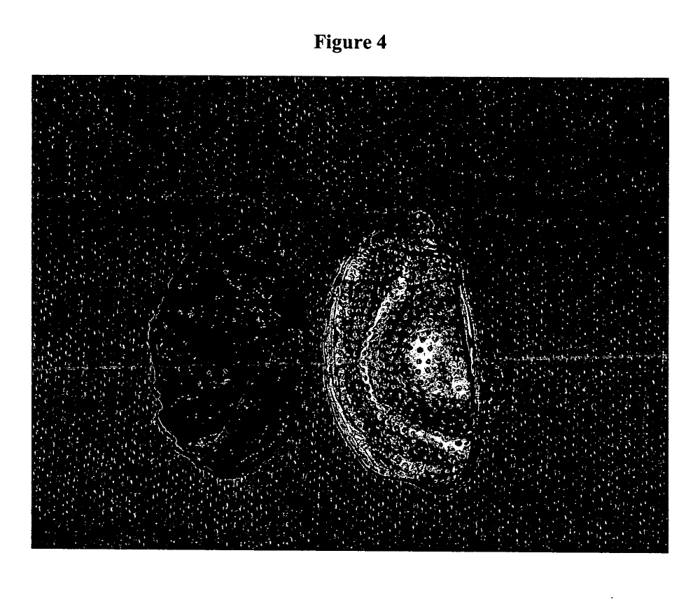

FIGS. 3A and 3B illustrate a cartilage ear in the opened mold, and removed from the mold, respectively. Regarding length of implantation, calcium alginate constructs were noted to be firmer, with enhanced anatomical details after 20 weeks of in vivo growth compared to the constructs removed at 12 to 16 weeks. There was also transition to small areas of bone formation noted in constructs removed at 20 weeks.

The constructs generated by using Pluronic F-127 were smaller in size due to tissue shrinkage, since much of the cartilage was noted on the exterior of the mold. Although the anatomical details internally were acceptable, the texture was more pliable than the alginate constructs. The constructs grown by using PGA demonstrated fibrous tissue only inside the mold and no cartilage outside of the mold was detectable. The control mold contained only small amount of fibrous tissue inside.

Histology

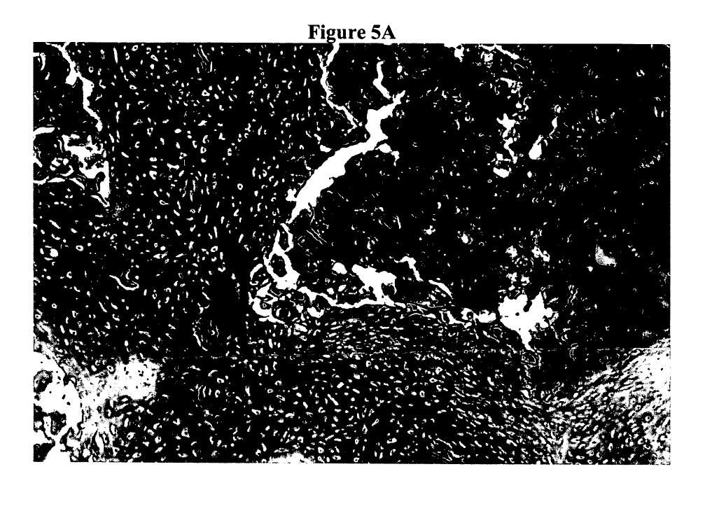

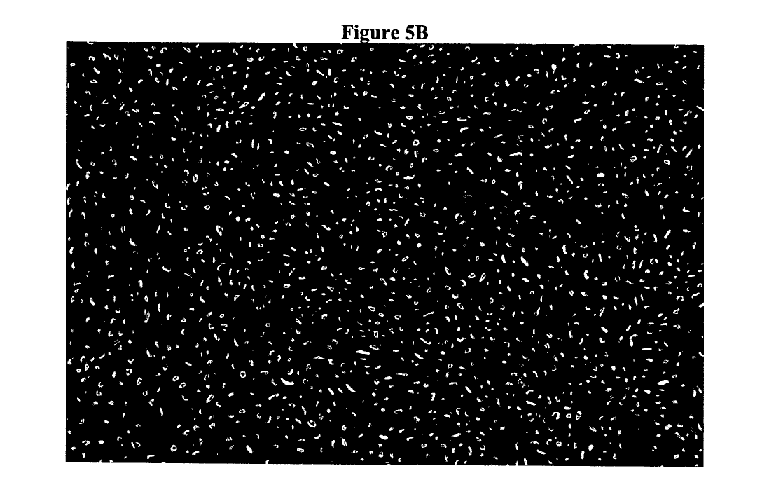

The constructs generated by the mixture of chondrocytes and calcium alginate demonstrated lobules of cartilage surrounded by broad bands of fibrous tissue (FIG. 5A). There were focal cystic areas containing asteroid materials and calcification. The tissue showed an irregular combination of cartilage with fibrous tissue. The Safranine-O stain was evenly positive in the areas of lobules of cartilaginous tissue suggestive of proteoglycan secretion. Areas of fibrous tissue were negative for this stain. The calcium alginate, possibly due to a thickened consistency, contained the cells inside the mold and resulted in an exact anatomical definition of the auricle. The tissue generated with alginate was approximately 60% cartilage; and alginate was still present after the 5 months of in vivo incubation. A lower concentration of alginate, and/or a longer incubation time, can be used to ensure that the alginate will be substantially removed from the construct by the time the mold is removed from incubation.

The tissue engineered cartilage grown from Pluronic F-127 demonstrated lobular cartilage (FIG. 5B). The cartilage was highly cellular with round to oval lacunae containing single nuclei. Occasional binucleate forms were also seen. The cytoplasm was abundant and contained condensed linear eosinophilic fragments of materials suggestive of elastin. Areas bordering the lobular cartilage showed flattened collagenous tissue suggestive of perichondrium. No inflammation was seen. No foreign body reaction was detectable. The tissue was essentially normal cartilage. Safranine-O stain of the same specimen demonstrated strong positivity throughout suggestive of proteoglycan presence. The fibrous perichondrium tissue was highlighted due to the absence of Safranine-O staining. Thus, the hydrogel Pluronic F-127 consistently produced the best histological quality of the final tissue engineered cartilage.

The constructs grown by using PGA displayed broad bands of collagenous tissue with intervening thin irregular strands of wavy fibrous tissue. Some areas appeared to show focal necrosis. The tissue was essentially fibrocollagenous debris. Safranine-O stain was negative for proteoglycan production.

The histology of the tissue inside the control mold showed fibrosis.

### OTHER EMBODIMENTS

A number of embodiments of the invention have been described. Nevertheless, it will be understood that various modifications may be made without departing from the spirit and scope of the invention.

1. A method of making a living tissue construct having a predetermined shape, the method comprising
obtaining a negative mold having a defined shape;
suspending isolated tissue precursor cells in a hydrogel to form a liquid hydrogel-precursor cell composition;
filling the liquid hydrogel-precursor cell composition into the mold;
implanting the filled mold into a living host for a time sufficient to enable the tissue precursor cells to form tissue;
removing the mold from the living host; and
removing the living tissue construct from the mold.

2. The method of claim 1, wherein the tissue precursor cells are chondrocytes, osteocytes, osteoblasts, or adipocytes, or a combination thereof.

3. The method of claim 1, wherein the tissue precursor cells are chondrocytes.

4. The method of claim 1, wherein the hydrogel is selected from the group consisting of alginate, chitosan, pluronic, collagen, and agarose.

5. The method of claim 1, wherein the hydrogel is alginate.

6. The method of claim 5, wherein the alginate concentration is from 0.5% to 8%.

7. The method of claim 5, wherein the alginate concentration is from 1% to 4%.

8. The method of claim 5, wherein the alginate concentration is approximately 2%.

9. The method of claim 1, further comprising inducing gel formation of the liquid hydrogel-precursor cell composition either before or after filling the composition into the mold.

10. The method of claim 9, wherein inducing gel formation comprises contacting the liquid hydrogel with a suitable concentration of a divalent cation.

11. The method of claim 9, wherein the divalent cation is Ca⁺⁺.

12. The method of claim 1, wherein the filled mold is incubated in the host for a period of 8 to 20 weeks.

13. The method of claim 1, wherein the negative mold is prepared using CAD/CAM or rapid prototyping.

14. The method of claim 1, wherein the filled mold is incubated in the host for a time sufficient for at least 50 percent of the hydrogel to be degraded and removed from the tissue construct within the mold.

15. A method of reconstructing an anatomical feature in a mammal, the method comprising
obtaining a suitable negative mold having a negative shape of the anatomical feature;
suspending isolated tissue precursor cells in a hydrogel to form a liquid hydrogel-precursor cell composition,
filling the liquid hydrogel-precursor cell composition into the mold;
inducing gel formation of the liquid hydrogel-precursor cell composition to form a living tissue construct;
implanting the filled mold into a living host for a time sufficient to enable the tissue precursor cells to form tissue;
removing the mold from the living host;
removing the tissue construct from the mold; and
implanting the tissue construct into the mammal.

16. An injection-molded living tissue construct made by the process of claim 1.

17. A method of reconstructing an anatomical feature in a mammal, the method comprising
obtaining a living tissue construct having the shape of the anatomical feature; and
implanting the tissue construct into the mammal, wherein the construct is prepared by the method of claim 1.

18. The method of claim 1, wherein the living tissue construct is shaped in the form of articular cartilage adjacent ajoint, a bone, a portion of a bone, or a bone defect.

19. The method of claim 1, wherein the hydrogel is selected from the group consisting of polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and sulfonated polymers.

20. The method of claim 1, wherein the tissue precursor cells are selected from the group consisting of epidermal cells, chondrocytes and other cells that form cartilage, macrophages, dermal cells, muscle cells, hair follicles, fibroblasts, organ cells, osteoblasts and other cells that form bone, endothelial cells, mucosal cells, pleural cells, ear canal cells, tympanic membrane cells, peritoneal cells, Schwann cells, corneal epithelial cells, gingiva cells, neural cells, neural stem cells, and tracheal epithelial cells.

21. The method of claim 1, wherein the tissue precursor cells are nervous system neural stem or progenitor cells.
